(19) United States
(12) Patent Application Publication
(10) Pub. No.: US 2020/0054534 A1
(43) Pub. Date: Feb. 20, 2020

(54) DRY COMPOSITION FOR REMOVING OILS FROM HAIR AND METHODS FOR FABRICATION OF DRY COMPOSITIONS

(71) Applicant: Mana Products, Inc., Long Island City, NY (US)

(72) Inventors: George Lambridis, Wayne, NJ (US); Julio Pina, Woodside, NY (US)

(21) Appl. No.: 16/151,735

(22) Filed: Oct. 4, 2018

Related U.S. Application Data
(60) Provisional application No. 62/567,938, filed on Oct. 4, 2017.

(51) Int. Cl. A61K 8/02; A61K 8/37; A61K 8/26; A61K 8/25; A61Q 5/00; A61Q 5/02

(52) U.S. Cl. CPC A61K 8/022; A61K 8/37; A61K 8/26; A61K 2800/592; A61Q 5/008; A61Q 5/02; A61K 2800/805; A61K 8/25

(57) ABSTRACT

A method of providing a treatment for cleaning and removing oils from hair includes providing a cosmetic that includes a blend of a powder phase including at least two absorbents selected from the group of mica, silica, starch, talc, kaolin and zeolite, and at least one emollient with a solvent phase. The blended powder and solvent phases are formed into a dry shampoo product adapted for manual application to the hair.

### BACKGROUND OF THE DISCLOSURE

### 1. Field of the Disclosure

The present disclosure relates to health and cosmetic treatment methods, and in particular, relates to dry shampoo powder compositions.

### 2. Description of the Related Art

Most hair cleaning products, such as shampoos, are offered to consumers in liquid or gel form. One of the disadvantages of such products is that use requires wetting of the hair, and a subsequent rinsing from the hair, and then drying of the hair. Although liquid hair products are effective for removing oil and dirt, these products often include surfactants that may damage hair or strip hair of color. Use can be inconvenient and may require other care to restore hair to a desired look.

As an alternative, various dry shampoos have been offered on the market for cleaning hair. Typically, dry shampoo is sold in loose powder or aerosol form. Although use of dry shampoo may be less involved, currently offered forms are difficult to apply efficiently when specific areas of hair are targeted. A form of dry shampoo that is easy to control and apply would be useful to many individuals that are on-the-go.

### SUMMARY OF THE DISCLOSURE

The present disclosure provides a method of providing a treatment for cleaning and removing oils from hair which comprises blending a powder phase including at least two absorbents selected from the group of mica, silica, starch, talc, kaolin and zeolite, and at least one emollient with a solvent phase. The blended powder and solvent phases are formed into a dry shampoo tablet adapted for manual and direct application to the hair.

In another embodiment, the present disclosure also provides a method of providing a treatment for deodorizing hair which comprises blending a solvent phase with a powder phase including a) at least two absorbents selected from the group of mica, silica, starch, and talc; and kaolin; b) zeolite; and c) at least one emollient. The blended powder and solvent phases are formed into a dry deodorizing tablet adapted for manual application to the hair.

In addition, the present disclosure provides a method of providing a treatment for hair which comprises blending a powder phase including at least two absorbents selected from the group of mica, silica, starch, talc, kaolin and zeolite, at least one emollient, and at least one colorant or highlighting component with a solvent phase. The blended powder and solvent phases are formed into a dry tablet adapted for manual application to the hair.

### BRIEF DESCRIPTION OF THE FIGURES

The features and advantages of the disclosure are apparent from the following description taken in conjunction with the accompanying drawings in which:

FIG. 1 is a perspective view of a cosmetic in a solid form;

FIG. 2 is a perspective view of a mold for fabricating the cosmetic of FIG. 1;

FIG. 3 is a view of a further embodiment in accordance with the disclosure.

### DETAILED DESCRIPTION OF THE DISCLOSURE

Disclosed herein are compositions of matter, products of manufacture that provide users with various forms of cosmetic materials (hereafter “cosmetic” or “cosmetics”). Examples of cosmetics that may be fabricated according to the techniques disclosed herein include dry shampoo, foundation, blush stick and eye shadow.

As an overview, techniques for fabricating the cosmetics include use of a mold. A composition for a particular type of cosmetic is disposed into an appropriately shaped mold in a slurry form. Generally, the slurry form includes a “powder phase” and a “solvent phase.” The ingredients of the powder phase may be varied according to the particular type of cosmetic. Likewise, ingredients of the solvent phase may be varied according to the particular type of cosmetic. Broadly, the solvent phase may include solvents such as water and may include other appropriate solvents to aid in the mixing and shaping of the cosmetic composition.

Examples of base compositions (i.e., the powder phase) for the cosmetic materials are provided in Table I. Included are compositions suited for use as dry shampoo, blush stick and eye shadow. Examples of solvent phase compositions for the cosmetic materials are provided in Table II. Included are compositions suited for use as the solvent phase for the dry shampoo, blush stick and eye shadow compositions of Table I.

TABLE I
Cosmetic Compositions for Mold

Range by weight percent

| Material | Low | High |
|---|---|---|
| Dry Shampoo | | |
| Mica | 10 | 60 |
| Silica | 5 | 40 |
| Corn starch | 5 | 20 |
| Rice starch | 5 | 20 |
| Talc | 1 | 10 |
| Kaolin (clay) | 1 | 10 |
| Zeolite | 1 | 5 |
| Isodecyl neopentanoate | 1 | 10 |
| Iron oxides | 5 | 30 |
| Preservatives | As needed | As needed |
| Fragrance | As needed | As needed |
| Blush | | |
| Mica | 10 | 60 |
| Silica | 5 | 20 |
| Corn starch | 5 | 20 |
| Talc | 1 | 10 |
| Lauroyl lysine | 1 | 5 |
| Isodecyl neopentanoate | 1 | 10 |
| Iron oxides | 5 | 15 |
| Preservatives | As needed | As needed |
| Fragrance | As needed | As needed |
| D&C red #7 lake | 5 | 10 |
| D&C red #6 | 5 | 10 |
| D&C red #28 lake | 1 | 5 |
| Eye Shadow | | |
| Mica | 10 | 60 |
| Silica | 5 | 20 |
| Corn starch | 5 | 20 |
| Talc | 1 | 10 |
| Lauroyl lysine | 1 | 5 |
| Isodecyl neopentanoate | 1 | 10 |
| Iron oxides | 5 | 30 |
| Preservatives | As needed | As needed |
| Fragrance | As needed | As needed |

TABLE II
Solvent Phase Compositions for Mold

Range by weight percent

| Material | Low | High |
|---|---|---|
| Dry Shampoo | | |
| Water | 90 | 100 |
| Glycerin | 2 | 10 |
| Carageenan | 0.1 | 1 |
| Xanthan gum | 0.1 | 1 |
| Preservative | As needed | As needed |
| Blush Stick | | |
| Water | 90 | 100 |
| Glycerin | 2 | 10 |
| Carageenan | 0.1 | 1 |
| Xanthan gum | 0.1 | 1 |
| Preservative | As needed | As needed |
| Eye Shadow Stick | | |
| Water | 90 | 100 |
| Glycerin | 2 | 10 |
| Carageenan | 0.1 | 1 |
| Xanthan gum | 0.1 | 1 |
| Preservative | As needed | As needed |

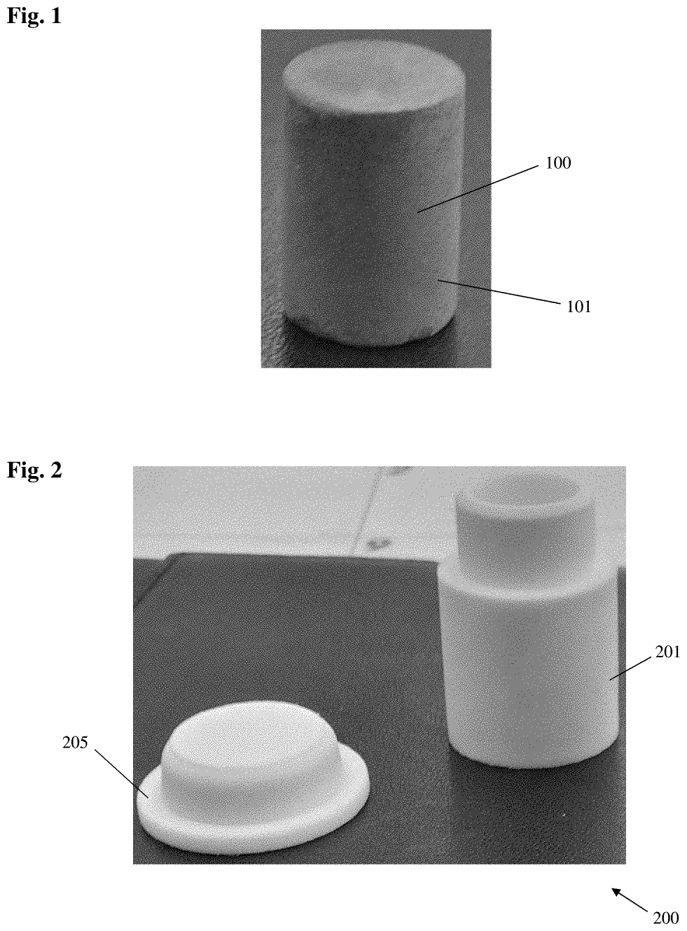

Referring now to FIG. 1, aspects of a cosmetic material 100, or simply a “cosmetic 100” are shown. In this example, the cosmetic 100 includes a dry shampoo composition that is provided as a shampoo stick 101. In this illustration, the shampoo stick 101 is separated from a dispenser and has the appearance of an elongated cylinder.

Referring now also to FIG. 2, aspects of an exemplary mold 200 for fabricating the shampoo stick 101 of FIG. 1 are shown. In this example, the mold 200 includes a cylindrically shaped body 201 with open ends (i.e., an annular cylinder). In one embodiment, the material is a thermoplastic material, such as a form of polyethylene (PE). As further examples, ultra-high molecular weight polyethylene (UHMWPE), high-density polyethylene (HDPE), polypropylene (PP), polytetrafluoroethylene (PTFE), and polyvinylidene fluoride (PVDF) may be used as the base materials for the mold 200. Still other materials include, without limitation, ethylene vinyl acetate (EVA), polyethersulfone (PES), polyurethane (PU) and PE/PP co-polymer.

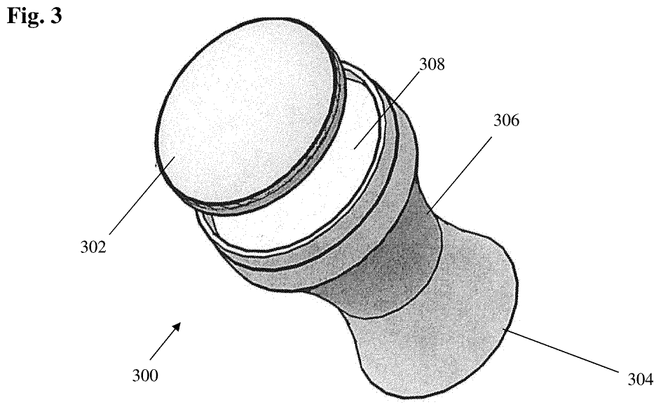

FIG. 3 is a perspective view of an embodiment of a dry hair treatment product 300 that includes a product such as the dry shampoo composition. In this example, the hair treatment product 300 includes a disc-shaped tablet (hereinafter referred to as the “shampoo tablet”) 302. In some embodiments the shampoo tablet may 302 be approximately 1 to 3 inches in diameter and 0.5 to 2.0 inches in thickness. The shampoo tablet 302 may also be formed in other sizes. In the depicted embodiment, an ergonomically-designed container 304, may be generally cylindrical, but may also include a narrowed middle or waist region 306 for manual handling, has at its top a disc-shaped platform space 308 in which the shampoo tablet 302 may be stored. The container 304 may also function as an applicator as the shampoo tablet 302 may be made to fit tightly within the platform 308 so that it does not fall out of the platform when the container is turned when applying the shampoo tablet to specific areas of hair. It is noted that the container shown in FIG. 3 is exemplary and that other containers having different shapes and functionality may be used. For example, in some embodiments, the platform 308 may be height-adjustable using mechanisms well known in the art.

Generally, and for some embodiments, the mica component of the powder phase, which may be the largest by weight percentage in a given composition imparts a dry texture to the shampoo tablet, and also imparts a natural shine to hair. The silica also provides texture and useful oil-absorbent properties, imparting smoothness. The modified starch, which is preferably a tapioca starch, but may also comprise corn and/or rice starch improves aesthetic effects by absorbing oily and greasy hair components. Talc and clay have oil and grease mitigation properties similar to the modified starch and can be used in addition to, or as a substitute for the starch. Zeolite is effective at capturing small particles and is particularly useful at reducing odors, e.g., odors arising from cosmetic products. Zeolite absorbs a variety of water-based and anhydrous compositions. Isodecyl neopentanoate acts as a binding ester and may be used to improve emolliency and the even distribution of the finished product, and may also improve aesthetic effects. In some embodiments, a silicon containing material such as dimethicone may be used as an emollient. Lauroyl lysine is an amino-acid based powder that acts as an anti-static agent and aids in the distribution of the other components.

In some implementations, the shampoo tablet 302 may be produced by the following exemplary process. First, the powder phase and the solvent phase are mixed well in a container, such as a kettle. The powder phase is mixed with the solvent phase according to a specific ratio from approximate 1:2 to 2:1 depending on desired properties. Once the phases are mixed into a homogeneous and uniform bulk phases (i.e., the “slurry”), the bulk phase is pumped and transferred to a feed nozzle and injected onto a ceramic or plastic substrate. The bulk phase material is then pressed into shaped tablets using a punch, while simultaneously a negative vacuum is created to extract most of the solvent. The pressed material is then then transferred onto trays which are then feed into a heater and heated at approximately 25 degrees Celsius to about 75 degrees Celsius (° C.), depending upon the solvent used, until the shampoo tablets 302 are adequately dry.

The resultant shampoo tablet 302 product has the texture of an emollient powder “cake.” The feel of the cake balances and combines the granularity of a powder with emollience and smoothness. The granularity increases the surface area of the cake product, providing improved absorbance, while the emollient properties improve aesthetic effects, such as shine and smoothness. As noted above, the shampoo tablet may be applied to specific areas of hair to clean and remove oils from hair fibers, and as a deodorant. Additionally, during production, colorant components may be added to the powder phase, and the resulting shampoo tablets may be used to color or add highlights to specific areas of hair where the tablet is applied.

Importantly, there is no need to rinse the hair after application of the dry shampoo disclosed herein as minimal residue is leftover, and as the hair remains dry.

Having thus introduced aspects of dry shampoo and a process for manufacture thereof, some additional embodiments and/or features are now provided.

In some embodiments, the mold, or portions of the mold, may also serve as a case for the finished cosmetic product. For example, once the manufacturing process has completed, branding may be applied to the body of the mold, such as in the form of a wrapper disposed thereover (not shown). In addition to carrying branding information, the wrapper may provide a moisture barrier to limit moisture intrusion into the cosmetic product.

Cosmetic products fabricated according to the techniques disclosed herein may be provided in a variety of forms (e.g., in varying lengths and/or widths).

Further, while referred to as being a “shampoo stick” in some embodiments, it should be recognized that the cosmetic composition and process for manufacture thereof may be useful for other purposes. For example, a similar process may be used to provide an “eye shadow stick.” More specifically, colors in the form of pigments and/or dyes may be added to the composition which is in turn loaded into a similar mold (albeit of differing proportions). Similarly, the stick cosmetic may be specially formulated and configured for use as foundation or blush.

The final form of the cosmetic need not be limited to stick form. That is, the solid form cosmetic may be provided in any geometry deemed appropriate.

Various other components may be included and called upon for providing for aspects of the teachings herein. For example, additional materials, combinations of materials and/or omission of materials may be used to provide for added embodiments that are within the scope of the teachings herein.

A variety of modifications of the teachings herein may be realized. Generally, modifications may be designed according to the needs of a user, designer, manufacturer or other similarly interested party. The modifications may be intended to meet a particular standard of performance considered important by that party.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. As used herein, the term “exemplary” is not intended to imply a superlative example. Rather, “exemplary” refers to an embodiment that is one of many possible embodiments.

Where a range of weight percentages of ingredients is described herein, the present disclosure contemplates that any value in increments of 0.1 weight percent between (and including) the endpoints of any such range can be practiced, wherein the total weight of the components of the overall composition total 100 weight percent.

While the disclosed embodiments have been described with reference to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A method of providing a treatment for cleaning and removing oils from hair, comprising:
blending a powder phase including at least two absorbents selected from the group of mica, silica, starch, talc, kaolin and zeolite, and at least one emollient, with a solvent phase; and
forming the blended powder and solvent phases into a dry shampoo tablet adapted for manual application to the hair.

2. The method of claim 1, wherein the step of forming the dry shampoo tablet includes molding and heating the blended powder and solvent phases.

3. The method of claim 1, wherein the dry shampoo tablet is formed in the shape of a disc having a diameter of between approximately 1 and 3 inches.

4. The method of claim 1, wherein the emollient includes isodecyl neopentanoate.

5. The method of claim 1, wherein the powder phase includes zeolite at 1 to 5 weight percentage.

6. The method of claim 1, wherein the powder phase further includes a fragrance component.

7. The method of claim 1, wherein the powder phase further includes a colorant component.

8. A method of providing a treatment for deodorizing hair, comprising:
blending a solvent phase with a powder phase including:
at least two absorbents selected from the group of mica, silica, starch, talc, and kaolin;
zeolite; and
at least one emollient; and
forming the blended powder and solvent phases into a dry deodorizing tablet adapted for manual application to the hair.

9. The method of claim 8, wherein the step of forming the dry shampoo tablet includes molding and heating the blended powder and solvent phases.

10. The method of claim 8, wherein the dry deodorizing tablet is formed in the shape of a disc having a diameter of between approximately 1 and 3 inches.

11. The method of claim 8, wherein the emollient includes isodecyl neopentanoate

12. The method of claim 8, wherein the zeolite form 1 to 5 weight percent of the powder phase.

13. A method of providing a treatment for hair, comprising:
blending a powder phase including at least two absorbents selected from the group of mica, silica, starch, talc, kaolin and zeolite, at least one emollient, and at least one colorant or highlighting component, with a solvent phase; and
forming the blended powder and solvent phases into a dry tablet adapted for manual application to the hair.

14. The method of claim 13, wherein the step of forming the dry shampoo tablet includes molding and heating the blended powder and solvent phases.

15. The method of claim 13, wherein the dry tablet is formed in the shape of a disc having a diameter of between approximately 1 and 3 inches.

16. The method of claim 13, wherein the emollient includes isodecyl neopentanoate.
